(19) United States
(12) Patent Application Publication
(10) Pub. No.: US 2023/0123462 A1
(43) Pub. Date: Apr. 20, 2023

(54) IN VITRO PROLIFERATION MEDIUM, IN VITRO CULTURE KIT AND IN VITRO PROLIFERATION CULTURE METHOD OF UMBILICAL CORD BLOOD (UCB)-DERIVED NATURAL KILLER (NK) CELLS

(71) Applicant: XINLU CELL BIOTECHNOLOGY (HAINAN) CO., LTD., HAIKOU CITY (CN)

(72) Inventors: Zhaoyong YANG, Beijing (CN); Yuanyuan JIN, Beijing (CN); Qiang HAN, Beijing (CN); Kun LIU, Beijing (CN); Wenzhuo YANG, Beijing (CN); Zhengyang SUN, Beijing (CN); Zhongbo WANG, Beijing (CN); Shuai FAN, Beijing (CN)

(21) Appl. No.: 17/671,312

(22) Filed: Feb. 14, 2022

(30) Foreign Application Priority Data
Oct. 18, 2021 (CN) 202111208066.6

(51) Int. Cl. C12N 5/0783

(52) U.S. Cl. CPC C12N 5/0646; C12N 2506/025; C12N 2501/2302; C12N 2501/2315

(57) ABSTRACT

The present disclosure provides an in vitro proliferation medium, an in vitro culture kit and an in vitro proliferation culture method of umbilical cord blood (UCB)-derived natural killer (NK) cells, and relates to the technical field of cell in vitro culture. In the present disclosure, the in vitro proliferation medium does not include animal serum to reduce immune responses when being used, with better safety. The in vitro proliferation medium is used to proliferate the UCB-derived NK cells in vitro, with simple and easy operations and low cost, and without coating, sorting and trophoblast cells. The medium does not include animal-derived ingredients, and has desirable safety and stability. The medium can be used for direct culture of peripheral blood mononuclear cells (PBMCs) isolated by Ficoll, and harvest more NK cells that meet clinical use standards.

### CROSS REFERENCE TO RELATED APPLICATION(S)

This patent application claims the benefit and priority of Chinese Patent Application No. 202111208066.6, filed on Oct. 18, 2021, the disclosure of which is incorporated by reference herein in its entirety as part of the present application.

### TECHNICAL FIELD

The present disclosure belongs to the technical field of cell culture in vitro, and specifically relates to an in vitro proliferation medium, an in vitro culture kit and an in vitro proliferation culture method of umbilical cord blood (UCB)-derived natural killer (NK) cells.

### BACKGROUND

Natural killer cells (NK cells) are effector cells of the natural immune system in human body, and are main functional cells for monitoring malignant pathological cells in vivo. NK cells, as a main force of the human body to fight tumors, recognize pathological cells by various activating and inhibitory receptors inherently expressed on a cell surface, play a first-line anti-infection and eliminate cells with malignant transformation, aging and damage. The NK cells are derived from many sources, including peripheral blood (PB), umbilical cord blood (UCB), induced pluripotent stem cells and embryonic stem cells. UCB-derived NK cells are regarded as a potential “off-the-shelf” product with many advantages.

However, a proliferation method of the NK cells in vitro is relatively complicated, and large-scale uses thereof lie first in simplification of the production process. Based on a proliferation and activation process of feeder cells, the NK cells have a relatively high proliferation multiple (thousands to tens of thousands of times), and a purity reaching about 90%; however, the presence of feeder cells increases the risk of clinical use and requires more product verification procedures. Based on a feeder cell-free proliferation system of cytokines and antibodies, the NK cells can have a proliferation multiple of thousands of times and a purity reaching about 70%, while avoiding the risk of using feeder cells; however, there are still significant individual differences in this production process, including cell activation and retention time in the body. Therefore, it is still a challenge for people to explore an NK cell production process with excellent NK cell purity, quantity, product stability, safety and process operability.

### SUMMARY OF THE INVENTION

In view of this, the purpose of the present disclosure is to provide an in vitro proliferation medium, an in vitro culture kit and an in vitro proliferation culture method of UCB-derived NK cells, which are simple, safe, stable and highly-versatile.

To achieve the objective of the present disclosure, the present disclosure provides the following technical solutions.

The present disclosure provides an in vitro proliferation medium of UCB-derived NK cells, where the in vitro proliferation medium uses a TheraPEAK™ X-VIVO™ 15 medium as a basic medium, and further comprises recombinant human interleukin-2 (rhIL-2).

Preferably, the rhIL-2 may have a working concentration of 200-2,000 IU/mL.

The present disclosure further provides an in vitro culture kit of UCB-derived NK cells, including the in vitro proliferation medium.

Preferably, the kit may further include an activation medium of the UCB-derived NK cells, where the activation medium uses a CTS™ AIM V™ SFM (RUO) medium as a basic medium, and further includes an activation factor; and the activation factor includes: the rhIL-2, recombinant human interleukin-15 (rhIL-15), StemRegenin 1 and recombinant human peroxiredoxin-5 (recombinant hPRDX5).

Preferably, in the activation medium, the rhIL-2 may have a working concentration of 200-2,000 IU/mL, the rhIL-15 may have a working concentration of 10-50 ng/ml, the StemRegenin 1 may have a working concentration of 1-10 μM, and the recombinant hPRDX5 may have a working concentration of 1-50 μM.

The present disclosure further provides an in vitro proliferation culture method of UCB-derived NK cells, including the following steps: activating mononuclear cells isolated from UCB, and conducting in vitro proliferation culture using the in vitro proliferation medium; where the in vitro proliferation culture is conducted is at 37° C., with 5% CO₂ in a saturated humidity environment.

Preferably, the activated mononuclear cells in the in vitro proliferation medium may have a concentration of (1-5)×10⁶ cells/mL.

Preferably, the in vitro proliferation culture may be conducted for 10-15 days; meanwhile, a fresh in vitro proliferation medium may be supplemented every 2-3 days, and a cell density may be adjusted to (1-5)×10⁶ cells/mL.

Preferably, a method for the activating may include: inoculating the mononuclear cells into the activation medium of the in vitro culture kit for activation.

Preferably, during the activating, the mononuclear cells may have an inoculation density of (1-5)×10⁶ cells/mL, and may be cultured at 37° C., with 5% CO₂ in a saturated humidity environment for 1-5 days.

The beneficial effects are as follows: in the present disclosure, the in vitro proliferation medium of UCB-derived NK cells does not include animal serum to reduce immune responses when being used, with better safety. The in vitro proliferation medium is used to proliferate the UCB-derived NK cells in vitro, with simple and easy operations and low cost, and without coating and sorting and trophoblast cells. The medium does not include animal-derived ingredients, and has desirable safety and stability. The medium can be used for direct culture of peripheral blood mononuclear cells (PBMCs) isolated by Ficoll, and harvest more NK cells that meet clinical use standards. The prepared NK cells have an expression rate of CD3⁻CD56⁺ cells of not less than 90%, can highly express activating receptors such as NKG2D, NKp30 and NKp46, and have a strong tumor killing activity in vitro.

### BRIEF DESCRIPTION OF THE DRAWINGS

FIG. 1 shows NK cell purities detected by flow cytometry before and after 14 d of culture; and

FIG. 2 shows expression of CD16 and NKG2D, NKp30 and NKp44 activating receptors by NK cells.

### DETAILED DESCRIPTION

The present disclosure provides an in vitro proliferation medium of UCB-derived NK cells, where the in vitro proliferation medium uses a TheraPEAK™ X-VIVO™ 15 medium as a basic medium, and further comprises rhIL-2.

In the present disclosure, the TheraPEAK™ X-VIVO™ 15 medium is purchased preferably from LONZA, USA. The rhIL-2 (purchased from Beijing SL Pharm Co., Ltd.) has a working concentration of preferably 200-2,000 IU/mL, more preferably 500-1,500 IU/mL, and most preferably 1,000 IU/mL.

The present disclosure further provides an in vitro culture kit of UCB-derived NK cells, including the in vitro proliferation medium.

In the present disclosure, the kit further includes preferably an activation medium of the UCB-derived NK cells, where the activation medium uses preferably a CTS™ AIM V™ SFM (RUO) medium as a basic medium, and further includes an activation factor; and the activation factor includes preferably: the rhIL-2, rhIL-15 (purchased from PeproTech), StemRegenin 1 (purchased from Selleck) and recombinant hPRDX5 (disclosed in CN110105442A).

In the present disclosure, the CTS™ AIM V™ SFM (RUO) medium is a serum-free lymphocyte medium, purchased preferably from Thermo Fisher Scientific.

In the activation medium of the present disclosure, the rhIL-2 has a working concentration of preferably 200-2,000 IU/mL, more preferably 500-1,500 IU/mL, and most preferably 1,000 IU/mL. The rhIL-15 has a working concentration of preferably 10-50 ng/ml, more preferably 20-30 ng/mL, and most preferably 25 ng/mL. The StemRegenin 1 has a working concentration of preferably 1-10 μM, more preferably 2 μM. The recombinant hPRDX5 has a working concentration of preferably 1-50 μM, more preferably 5-20 μM, and most preferably 10 μM.

The present disclosure further provides an in vitro proliferation culture method of UCB-derived NK cells, including the following steps: activating mononuclear cells isolated from UCB, and conducting in vitro proliferation culture using the in vitro proliferation medium; where

the in vitro proliferation culture is conducted is at 37° C., with 5% CO₂ in a saturated humidity environment.

In the present disclosure, there is no specific limitation on a method for isolating the mononuclear cells, and the isolating is preferably conducted by Ficoll. A method for the activating includes preferably: inoculating the mononuclear cells into the activation medium of the in vitro culture kit for activation; during the activating, the mononuclear cells have an inoculation density of preferably (1-5)×10⁶ cells/mL, more preferably 2×10⁶ cells/mL, and are cultured at 37° C., with 5% CO₂ in a saturated humidity environment for 1-5 days.

In the present disclosure, the mononuclear cells are inoculated preferably into the CTS™ AIM V™ SFM (RUO) medium, the activation factor is added to reach the working concentration to conduct the activation for 1-5 days, and cell sap is collected.

In the present disclosure, the activated mononuclear cells are inoculated into an in vitro proliferation medium for in vitro proliferation culture; preferably into a TheraPEAK™ X-VIVO™ 15 medium, with an inoculation density of preferably (1-5)×10⁶ cells/mL, more preferably 2×10⁶ cells/mL; and the rhIL-2 is added to reaching the working concentration. The in vitro proliferation culture are conducted for preferably 10-15 days; meanwhile, a fresh in vitro proliferation medium is supplemented preferably every 2-3 days, and a cell density is adjusted to preferably (1-5)×10⁶ cells/mL to harvest the UCB-derived NK cells.

The NK cells prepared by the in vitro proliferation culture method have an expression rate of CD3⁻ CD56⁺ cells of not less than 90%, can highly express activating receptors such as NKG2D, NKp30 and NKp46, and have a strong tumor killing activity in vitro.

The in vitro proliferation medium, the in vitro culture kit and the in vitro proliferation culture method of UCB-derived NK cells provided by the present disclosure will be described in detail below with reference to examples, but these examples should not be construed as limiting the protection scope of the present disclosure.

### EXAMPLE 1

In Vitro Proliferation Culture Of UCB-Derived Nk Cells

1. Isolation of UCB-Derived Mononuclear Cells

(1) A blood bag containing UCB was sterilized, and the UCB was aspirated and added to a 50 mL centrifuge tube for centrifugation (at 2,000 r/min for 20 min, with a rise rate of 5 and a fall rate of 5).

(2) After the centrifugation was completed, an upper layer of plasma was discarded, and cell layers between the plasma and red blood cells were retained. The UCB in the centrifuge tube was resuspended with an equal amount of a phosphate-buffered saline (PBS) solution. Resuspended UCB was mixed well, and a lymphocyte isolation solution (a Ficoll-Hypaque solution, known as Ficoll, purchased from Tianjin HaoYang Biological Manufacture Co., Ltd.) at half volume of the resuspended UCB was added to a new 50 mL centrifuge tube. The resuspended UCB was slowly added on a surface of the Ficoll to keep a contact surface stable. The centrifuge tube was centrifuged (at 2,000 r/min for 20 min, with a rise rate of 2 and a fall rate of 2).

(3) After centrifugation, a white membrane between a Ficoll layer and a plasma layer could be clearly seen, namely the mononuclear cells. All the white membrane in the middle layer was pipetted through a pipette, placed in a new centrifuge tube, the cells were diluted with a PBS solution to a volume of 50 mL, centrifuged (at 1500 r/min for 5 min), and washed with PBS repeatedly for 3 times.

2. Activation Culture of NK Cells

Activation culture of UCB-derived NK cells: a serum-free lymphocyte medium CTS™ AIM V™ SFM (RUO) (purchased from Thermo Fisher Scientific) was used, a density of UCB-derived mononuclear cells was adjusted to 2×10⁶ cells/mL, 1000 IU /mL rhIL-2, 25 ng/ml rhIL-15, 2 μM of StemRegenin 1 and 10 μM of recombinant hPRDX5 were added, cells were cultured at 37° C., with 5% CO₂ in a saturated humidity environment for 5 days, and cell sap was collected.

3. Proliferation Culture of NK Cells

Proliferation culture of UCB-derived NK cells: cells were obtained from an activated cell sap, a TheraPEAK™ X-VIVO™ 15 cell medium (purchased from LONZA, USA) was used, a cell density was adjusted to 2×10⁶ cells/mL, 1000 IU/mL rhIL-2 was added, the cells were cultured at 37° C., with 5% CO₂ in a saturated humidity environment for 10 days, where a fresh medium containing rhIL-2 was supplemented every 2 days and the cell density was adjusted to 2×10⁶ cells/mL, to harvest the UCB-derived NK cells.

### EXAMPLE 2

Detection of UCB-Derived NK Cells

1. In vitro proliferation culture was conducted on UCB-derived mononuclear cells using the method of Example 1, and cell counting was conducted with an automatic cell counter (Countstar). The results are shown in Table 1 and Table 2, showing that the UCB-derived cells expand by an average of 305.13 times within 14 days, and expand by an average of 5,767.37 times in the same period using the in vitro culture kit and the in vitro proliferation culture method of the present disclosure.

TABLE 1
Total number and proliferation multiple
of UCB-derived cells (20 ml of UCB)

| | Sample 1 | Sample 2 | Sample 3 |
|---|---|---|---|
| Total number of UCB-derived | 2.03 | 1.85 | 2.14 |
| mononuclear cells at day 0 (×10⁷) | | | |
| Total number of proliferated | 598 | 622 | 609 |
| cells at day 14 (×10⁷) | | | |
| Total number of cells proliferation | 294.58 | 336.22 | 284.58 |
| multiple | | | |

TABLE 2
Absolute number and proliferation multiple
of UCB-derived NK cells (20 ml of UCB)

| | Sample 1 | Sample 2 | Sample 3 |
|---|---|---|---|
| Absolute number of UCB-derived | 1.05 | 1.01 | 1.01 |
| NK cells at day 0 (×10⁶) | | | |
| Absolute number of UCB-derived | 5856.2 | 6002.9 | 5839.1 |
| NK cells at day 14 (×10⁶) | | | |
| Proliferation multiple of UCB- | 5577.3 | 5943.5 | 5781.3 |
| derived NK cells | | | |

2. Detection of NK Cell Purity by Flow Cytometry

The results are shown in Table 3. By the in vitro culture kit and the in vitro proliferation culture method of the present disclosure, after 14 days of in vitro proliferation culture, the UCB-derived NK cells can have a purity of reaching 96.77% on average.

TABLE 3
Purity of UCB-derived NK cells

| | Sample 1 | Sample 2 | Sample 3 |
|---|---|---|---|
| Purity of UCB-derived NK cells | 5.18% | 5.45% | 4.47% |
| at day 0 | | | |
| Purity of UCB-derived NK cells | 97.93% | 96.51% | 95.88% |
| at day 14 | | | |

3. Phenotype Identification of NK Cells

A density of NK cells was counted, 1×10⁶ cells were centrifuged at 1,000 rpm for 5 min, supernatant was discarded, the cells were resuspended in 2 ml of PBS, and supernatant was discarded by centrifugation. The cells were resuspended in PBS, control tubes and test tubes were set up, and CD56, CD3, CD16, NKG2D, NKp44 and NKp30 antibodies were added, respectively (the antibodies were purchased from Biolegend). The cells were incubated for 20 min at 4° C. in the dark, and 2 ml of PBS was added to wash the cells to remove excess antibodies. The cells were resuspended with 200 μl of PBS, and detected by a flow cytometer.

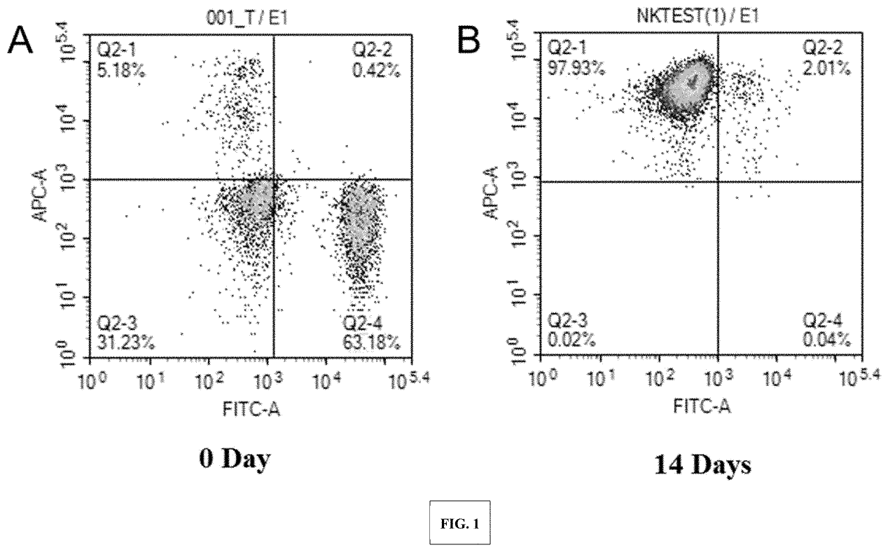

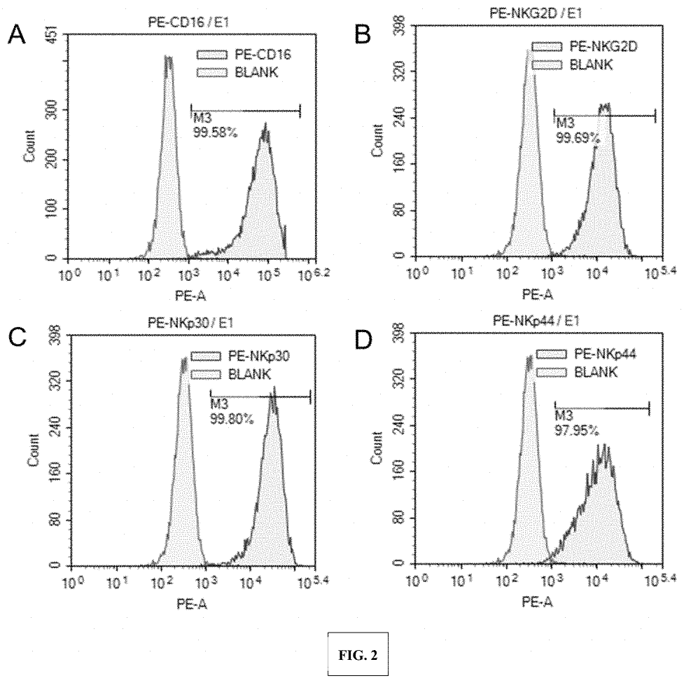

The phenotype of NK cells is CD3⁻ CD56⁺; after mononuclear cells are induced and cultured, CD3⁻ CD56⁺ cells increase from 5.18% before culture to 97.93% after culture, as shown in FIG. 1. In FIG. 1, A is flow cytometry test results of mononuclear cells before culture, and B is flow cytometry test results of mononuclear cells after 14 days of culture. The results indicate that mononuclear cells have basically been induced to form NK cells. In addition, almost all NK cells express CD16 and the activating receptors NKp44, NKp30 and NKG2D (A, B, C and D in FIG. 2).

4. Determine of Killing Activity of UCB-Derived NK Cells by Lactate Dehydrogenase (LDH) Method

The killing activity of NK cells against K562 and HGC-27 was detected using an LDH release method. Through preliminary experiments, an optimal cell volume of target cells was determined to be 1×10⁴ cells/well; after adjusting the cell concentration, 100 μl of cell suspension diluted with a medium was drawn to a 96-well plate. After overnight pre-culture, a new 100 μl of medium was changed for subsequent operations. The concentration of NK cells was adjusted according to different effector cell-target cell ratios; 100 μl of a medium containing NK cells was added to the corresponding wells, the cells were cultured in a 37° C. incubator with CO₂ for 3.5 h, 20 μl of a Lysis Buffer was added to high control wells, 20 μl of medium was added to low control wells and background blank wells, and the cells were incubated in a 37° C. incubator with CO₂ for 30 min. The 96-well plate was centrifuged for 2 min (250×g) to pellet suspended cells. 100 μl of supernatant was pipetted from each well into a new 96-well plate. After adding 100 μl of a Working Solution to each well, the cells were incubated for 20 min at room temperature in the dark. After adding 50 μl of a Stop Solution to each well, an absorbance at 490 nm was immediately measured with a microplate reader. Cell damage rate (%)=[(A−C)/(B−C)]×100, three replicate holes were made for each hole, and an average value was adopted; where,

A: An absorbance of samples (sample hole - sample blank hole);

B: An absorbance of high control (high control well - high control blank well); and

C: An absorbance of low control (low control well - background blank well).

The results are shown in Table 4.

TABLE 4
Killing ability of UCB-derived NK cells to K562 and HGC-27

| | K562 | HGC-27 |
|---|---|---|
| Effector cell-target cell ratio (4:1) | 87% | 100% |
| Effector cell-target cell ratio (2:1) | 56% | 72% |
| Effector cell-target cell ratio (1:1) | 30% | 56% |
| Effector cell-target cell ratio (1:2) | 14% | 30% |

The foregoing are merely descriptions of preferred examples of the present disclosure. It should be noted that several improvements and modifications can be made by a person of ordinary skill in the art without departing from the principle of the present disclosure, and these improvements and modifications shall also be deemed as falling within the protection scope of the present disclosure.

What is claimed is:

1. An in vitro proliferation medium of umbilical cord blood (UCB)-derived natural killer (NK) cells, wherein the in vitro proliferation medium uses a TheraPEAK™ X-VIVO™ 15 medium as a basic medium, and further comprises recombinant human interleukin-2 (rhIL-2).

2. The in vitro proliferation medium according to claim 1, wherein the rhIL-2 has a working concentration of 200-2,000 IU/mL.

3. An in vitro culture kit of UCB-derived NK cells, comprising the in vitro proliferation medium according to claim 1.

4. The in vitro culture kit according to claim 3, further comprising an activation medium of the UCB-derived NK cells, wherein the activation medium uses a CTS™ AIM V™ SFM (RUO) medium as a basic medium, and further comprises an activation factor; and the activation factor comprises: the rhIL-2, recombinant human interleukin-15 (rhIL-15), StemRegenin 1 and recombinant human peroxiredoxin-5 (recombinant hPRDX5).

5. The in vitro culture kit according to claim 4, wherein in the activation medium, the rhIL-2 has a working concentration of 200-2,000 IU/mL, the rhIL-15 has a working concentration of 10-50 ng/ml, the StemRegenin 1 has a working concentration of 1-10 μM, and the recombinant hPRDX5 has a working concentration of 1-50 μM.

6. An in vitro proliferation culture method of UCB-derived NK cells, comprising the following steps:
activating mononuclear cells isolated from UCB, and conducting in vitro proliferation culture using the in vitro proliferation medium according to claim 1; wherein
the in vitro proliferation culture is conducted is at 37° C., with 5% CO₂ in a saturated humidity environment.

7. The in vitro proliferation culture method according to claim 6, wherein the activated mononuclear cells in the in vitro proliferation medium has a concentration of (1-5)×10⁶ cells/mL.

8. The in vitro proliferation culture method according to claim 6, wherein the in vitro proliferation culture is conducted for 10-15 d; meanwhile, a fresh in vitro proliferation medium is supplemented every 2-3 days, and a cell density is adjusted to (1-5)×10⁶ cells/mL.

9. The in vitro proliferation culture method according to claim 7, wherein the in vitro proliferation culture is conducted for 10-15 days; meanwhile, a fresh in vitro proliferation medium is supplemented every 2-3 days, and a cell density is adjusted to (1-5)×10⁶ cells/mL.

10. The in vitro proliferation culture method according to claim 6, wherein a method for the activating comprises: inoculating the mononuclear cells into the activation medium of the in vitro culture kit according to claim 4 for activation.

11. The in vitro proliferation culture method according to claim 10, wherein during the activating, the mononuclear cells have an inoculation density of (1-5)×10⁶ cells/mL, and are cultured at 37° C., with 5% CO₂ in a saturated humidity environment for 1-5 days.

12. The in vitro proliferation culture method according to claim 6, wherein a method for the activating comprises: inoculating the mononuclear cells into the activation medium of the in vitro culture kit according to claim 5 for activation.

13. The in vitro proliferation culture method according to claim 12, wherein during the activating, the mononuclear cells have an inoculation density of (1-5)×10⁶ cells/mL, and are cultured at 37° C., with 5% CO₂ in a saturated humidity environment for 1-5 days.
